(19) United States
(12) Patent Application Publication
(10) Pub. No.: US 2011/0184469 A1
(43) Pub. Date: Jul. 28, 2011

(54) SET SCREW ALIGNMENT TOOL

(75) Inventors: Rodney R. Ballard, Lakeland, TN (US); Michael Allen Dupree, Munford, TN (US); Jesse Moore, Germantown, TN (US)

(73) Assignee: WARSAW ORTHOPEDIC, INC., Warsaw, IN (US)

(21) Appl. No.: 12/695,946

(22) Filed: Jan. 28, 2010

(51) Int. Cl. A61B 17/88; A61B 17/56

(52) U.S. Cl. 606/279; 606/86 A

(57) ABSTRACT

A surgical instrument for reducing a rod toward a bone fastener and securing it thereto with a set screw having a cylindrical fastener engaging member extending between a proximal end and a distal end. The distal portion of said fastener engaging member being adapted to receive a portion of the rod therein and being further adapted to engage the bone fastener. The instrument further being provided with a coaxial set screw installation member extending between a proximal end and a distal end. The distal end of the set screw installation member being adapted to receive and hold the set screw securely within its distal end. The fastener engaging member and coaxial set screw installation member being operable together to reduce the rod distally into the bone fastener and then rotationally attach the set screw into the bone fastener while holding the rod in contact with the bone fastener.

### FIELD OF THE INVENTION

The present invention relates generally to instruments used to manipulate orthopedic implants. More particularly, the invention relates to the manipulation of bone anchoring elements positioned in the spine and longitudinal members interconnecting those elements. And, while the present invention in useful in spinal surgery, it may find use in other areas of medicine as well.

### BACKGROUND

In the field of orthopedic surgery, and particularly spinal surgery, it is well known to correct an injury, malfunction, or other defect by use of an implanted rod affixed to the body part to be corrected. For example, rod systems have been developed for correcting the position of and stabilizing the spine, and for facilitating fusion at various levels of the spine. In one such system, the rod is disposed longitudinally along a length of the spine. The rod is preferably bent, either prior to or during surgery, to correspond to the normal curvature of the spine in the particular region being instrumented, or to such other curvature as the surgeon may deem appropriate to correct the defect. For example, the rod can be bent to form a normal kyphotic curvature for the thoracic region of the spine, or to form a normal lordotic curvature for the lumbar region. The rod is engaged to a number of fixation elements fixed to or engaged with the vertebrae along the segment of the spinal column.

A variety of fixation elements can be provided that are configured to engage the vertebrae. For instance, one such fixation element is a laminar hook, configured to engage a lamina of the vertebra. Another typical fixation element is a spinal screw which can be threaded into a pedicle or other portion of the of vertebral bone. Examples of such spinal screws are seen in U.S. Pat. No. 5,005,562 to Cotrel, the disclosure of which is incorporated herein by reference. An alternative type of fixation element is a multi-axial bone screw, such as that disclosed in U.S. Pat. Nos. 5,797,911 and 5,879,350 to Sherman et al., each of which is also incorporated herein by reference in its entirety. Further types of bone screws, hooks, bolts, or other fixation elements are known in the art.

In one typical spinal procedure, an elongated implant (e.g. a rod) is coupled to two or more fixation elements (e.g. bone screws) that are affixed along the spinal column. Often times, such an elongated implant and respective fixation elements are affixed along both sides of the spinous process in a parallel fashion. The bone screws are first threaded into a portion of several vertebral bodies, such as the pedicles of these vertebrae. The rod is coupled to the bone screws to provide corrective and stabilizing forces to the spine. Affixing a rod to a bone screw generally requires the rod to be in close adjacent position or in contact with the screw. For example, with respect to bone screws as disclosed in the above mentioned patents, a rod and an implanted screw must be moved with respect to each other so that the rod occupies space within a channel or other opening in the screw. The rod is then coupled to the implanted bone screw using a set screw, plug or other appropriate fastener. The process of placing a rod within or adjacent to an implanted fixation element so that they can be coupled together is termed “reducing” the rod.

Rod reduction is commonly performed by a surgeon using his or her hands and/or rigid tools such as pliers, levers, or other instrumentation adaptable to create the necessary pushing and/or pulling forces on the implanted screw and rod. And various attempts in the prior art have been made in providing rod reducing instruments, such as described in U.S. Pat. No. 6,036,692 to Burel et al.; and U.S. Pat. No. 5,910,141 to Morrison et al.; each of which is incorporated herein by reference in its entirety. However, needs remain in the industry for rod reducing instruments that can be used efficiently, safely and securely in rod reduction procedures and for rod reduction instruments that can be used in both minimally invasive and open surgical approaches to the site of rod attachment.

### SUMMARY

According to one aspect, instrumentation is provided for rod reduction during spinal surgery that is efficient and convenient to use. The instruments eliminate that need for direct application of manual force to a rod to position it in a desired location relative to a fastener. The instruments can also facilitate attachment of the rod to the fastener, and have application in both open surgical procedures and minimally invasive surgical procedures.

According to another aspect, instrumentation is provided for rod reduction during spinal surgery wherein the rod reduction instrument performs several functions while reducing the rod into the channel of the fastener. It is desirable for the instrumentation to first reduce the rod into an appropriate position within the channel of the fastener. The instrumentation should also allow for the use of a rod forceps or “rocker” tool at the same time, such tool being known in the art as disclosed in U.S. Pat. No. 6,036,692 to Burel et al. as disclosed above.

According to another aspect, the instrumentation should be able to hold an appropriate set screw or other closure device to capture the rod within the channel of the fastener. The set screw would typically be a break off set screw such that upon capturing the rod within the channel the back end of the set screw would break off from the threaded portion which would remain in the channel of the fastener. The broken off portion of the set screw would remain captured within the instrumentation such that it would not fall out of the instrumentation into the open surgical site.

According to yet another aspect, it would be desirable that the instrumentation would grip the head of the fastener is such a manner that would assist aligning the set screw and fastener and in preventing cross threading of the set screw within the channel of the fastener. Additionally the instrument would grip the head of the fastener to assist in preventing head splay while threading the set screw within the channel of the fastener. Finally, it would be desirable for the instrumentation to be provided with a counter-torque feature to allow for significant rotational forces to be applied to the set screw without such significant rotational forces being applied to the head of the fastener.

Aspects, objects, advantages, features, embodiments, and benefits of the present invention will be evident upon consideration of the following written description and the accompanying figures, which illustrate embodiments of the invention.

### BRIEF DESCRIPTION OF THE FIGURES

FIG. 1 is a front elevational view of a rod reducer instrument in accordance with one embodiment of the present invention shown in an at rest position.

FIG. 2 is a cross-sectional view of the rod reducer instrument illustrated in FIG. 1, as taken along line II-II of FIG. 1.

FIG. 2A is a cross-sectional view of a blown up portion of the rod reducer instrument illustrated in FIG. 2.

FIG. 3 is a perspective view of the distal tip of the rod reducer instrument shown in relation to a fastener, rod element and set screw.

FIG. 4 is side perspective view of the rod reducer instrument in partial cross section shown in close proximity with a fastener, rod element, and rod rocker tool.

FIG. 5 is a side perspective view of the rod reducer instrument in partial cross section shown in operation with a fastener, rod element, and rod rocker tool.

FIG. 6 is a side perspective view of the rod reducer instrument in partial cross section shown separated from the fastener, rod element and rod rocker tool with the set screw shown in place within the head of the fastener.

FIG. 7 is a cross sectional view of a portion of the rod reducer instrument shown in connection with the fastener and rod element.

FIG. 8 is a cross sectional view of a portion of the rod reducer instrument shown removed from the fastener and rod element with the set screw shown in place holding the rod securely within the head of the fastener.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation on the scope of the invention is intended. Any alterations and further modifications in the illustrated devices and described methods and further applications of the principles of the invention as disclosed herein are contemplated as would normally occur to one skilled in the art to which the invention relates.

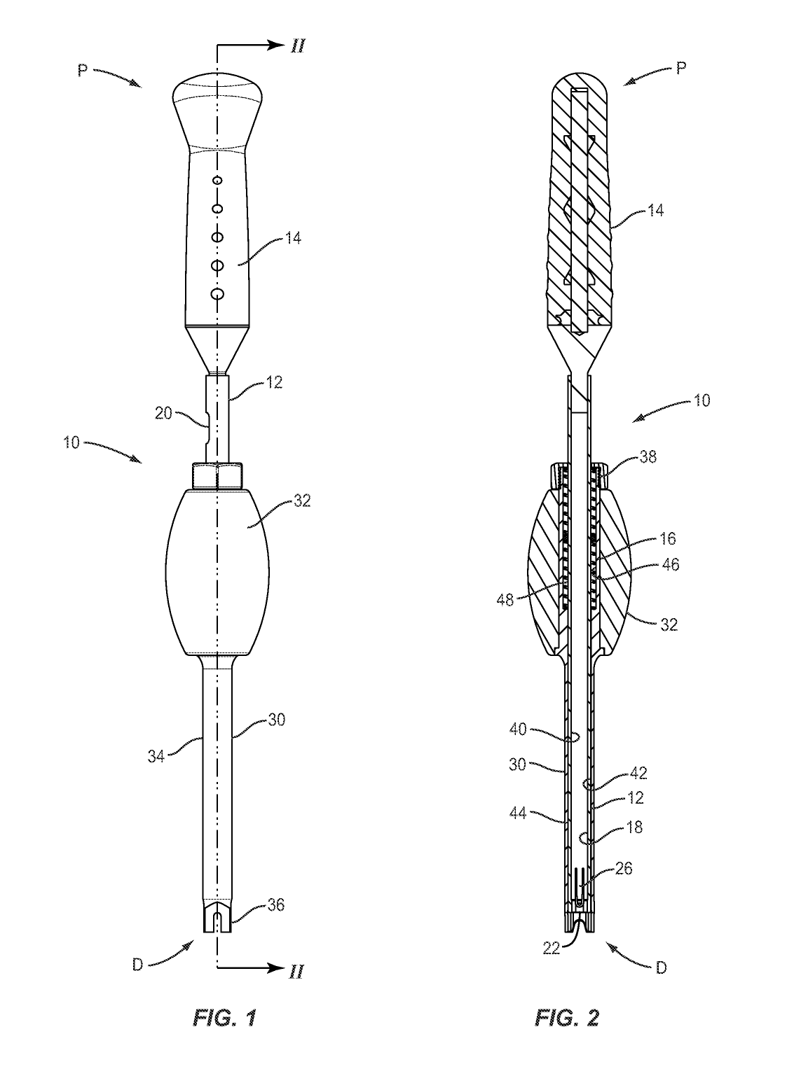

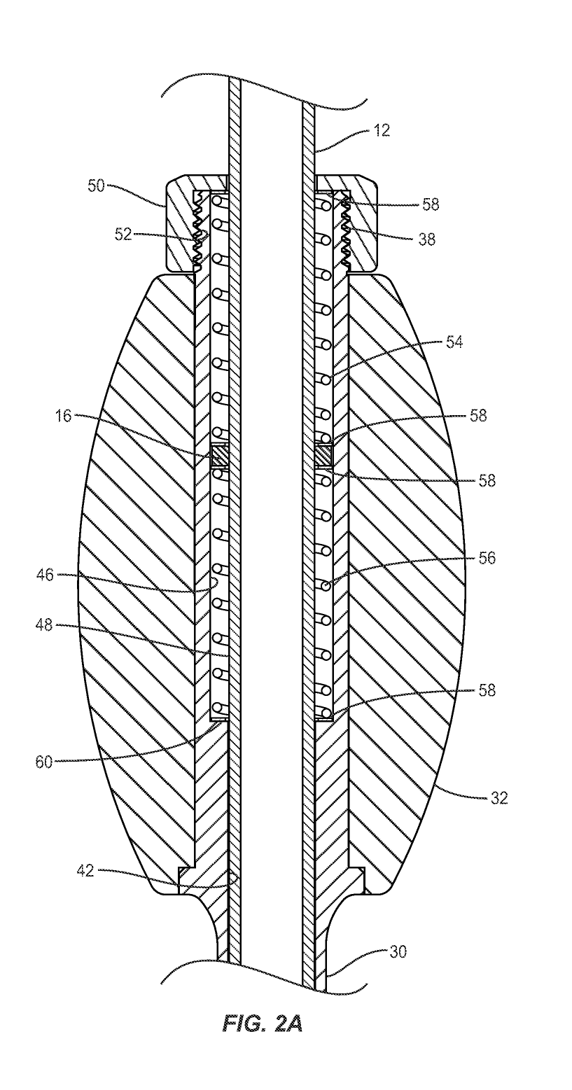

Referring to FIGS. 1 and 2, shown therein is one form of a rod reducer 10. Rod reducer 10 has a distal end D which would be positioned adjacent a pedicle screw and elongated rod being implanted within the body of a patient. Rod reducer 10 has a proximal end P which would be positioned outside of a patient's body. The rod reducer 10 has a first or inner shaft 12 having an enlarged handle 14 on its proximal end P. The enlarged handle 14 is designed for easy gripping by a surgeon user and may be provided with a rubberized gripping surface to allow the surgeon user to apply either axial pushing force or rotational force on the first shaft 12 to operate the rod reducer. The handle 14 may also have a roughened surface or be otherwise treated to allow for ease of gripping, pushing or rotation of the first shaft 12. The first shaft 12 extends longitudinally from its proximal end P to a distal end D. The first shaft 12 is of a generally uniform outer diameter with the exception of an enlarged portion 16 provided thereon which portion will be discussed in greater detail in relation to FIG. 2A, below. The first shaft 12 is provided with an axial bore 18 extending from its distal end D to a position adjacent the enlarged handle 14. An opening 20 is provided on first shaft 12 communicating with the proximal end of axial bore 18. A distal tip 22 of first shaft 12 is provided with an internal hexagonal configuration 24 which would match the outer configuration of a typical set screw for use with a spinal screw connector, discussed in greater detail below. The distal tip 22 of first shaft 12 is also provided with a pair of fingers 26 for grabbing the set screw as discussed further below.

Still referring to FIGS. 1 and 2, the rod reducer 10 is further provided with a second or outer shaft 30 having an enlarged knob 32 and an elongated shaft 34 extending distally from the knob 32. The shaft 34 is provided with a terminal distal end 36 which is configured to mate with the head of a screw to be implanted within the body, further discussed below. The outer shaft 30 is provided with an externally threaded portion 38 at its proximal end. The outer shaft 30 is provided with an axial bore 40. The axial bore 40 has a first inner diameter 42 which is slightly larger than the outer diameter 44 of the inner or first shaft 12, and a second inner diameter 46 which is slightly larger than the outer diameter 48 of enlarged portion 16 of the inner or first shaft 12. The enlarged knob 32 is designed for easy gripping by the surgeon user and may be provided with a metallic surface, a non metallic surface, or a rubberized gripping surface to allow the surgeon user to apply axial force to the outer shaft 30. The knob surface may also be roughened or otherwise treated to allow the surgeon user to easily grip the outer shaft 30 for axial movement thereof.

Referring now to FIG. 2A, the details of the connection of the inner and outer shafts 12 and 30 and shown in greater detail. A nut 50 is provided about the inner shaft 12. The nut 12 is positioned proximally from the enlarged portion 16 of shaft 12 and distally to the enlarged handle 14. The nut 50 can be positioned about the shaft 12 before the shaft is soldered or otherwise attached to enlarged proximal handle 14. The nut 50 has internal threads 52 open in a distal direction for attachment to the externally threaded portion 38 of outer shaft 30. Proximal and distal springs 54 and 56, respectively, are positioned adjacent the enlarged portion 16 and washers 58 may be positioned about the inner shaft 12 immediately adjacent the ends of the proximal and distal springs 54 and 56. Referring to FIG. 2A, the second or outer shaft 30 is shown positioned about the first or inner shaft 12 such that the proximal and distal springs 54 and 56 and washers 58 are captured within the radial space defined by the outer diameter 44 of shaft 12 and the second inner diameter 46 of the axial bore 40 of outer shaft 30 and the longitudinal space defined by the nut 50 when threaded about the externally threaded portion 38 of the outer shaft 30 and the distal end 60 of the second inner diameter portion 46 of axial bore 40. The proximal and distal springs 54 and 56 act to provide a balanced or at rest position of the outer shaft 30 about the inner shaft 12. The outer shaft 30 can be moved rotationally about inner shaft 12 and can also be moved axially with respect to shaft 12 at least as far as the springs 54 and 56 will allow. If the surgeon user lets go of the outer shaft 30 the springs 54 and 56 will act to axially move the outer shaft 30 back to its balanced or at rest position about inner shaft 12.

Referring back to FIGS. 1 and 2, the outer shaft 30 and inner shaft 12 are shown attached together in the balanced or at rest position as discussed above. When the inner and outer shafts 12 and 30 are shown in the balanced position, the distal end 36 of outer shaft 30 extends beyond the distal tip 22 of the inner shaft 12. The opening 20 of inner shaft 12 is also visible in a position proximal to the nut 50 attached to the outer shaft 30. If the outer shaft 30 is pulled back in a proximal direction with respect to the inner shaft 12, or the inner shaft 12 is pushed forward in a distal direction with respect to the outer shaft 30, the distal tip 22 on the inner shaft 12 will extend distally beyond the distal end 36 of the outer shaft 30.

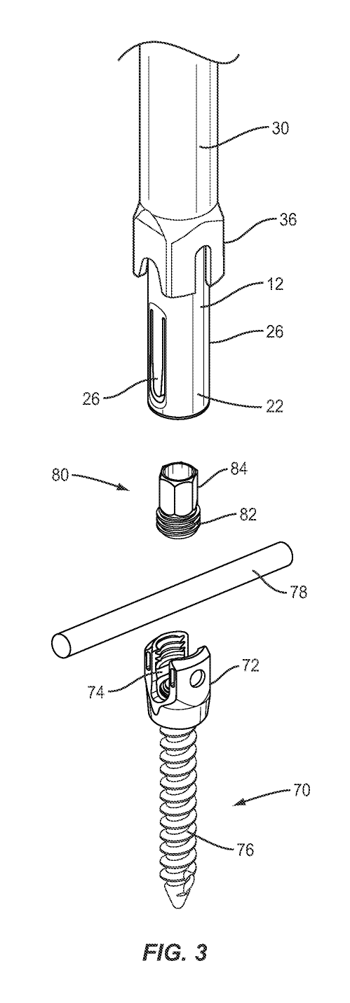

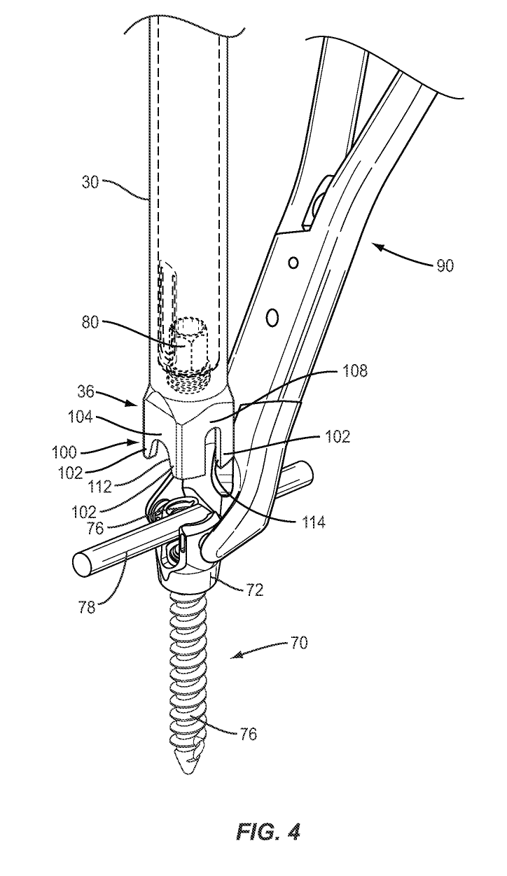

Referring now to FIG. 3, a typical pedicle screw 70 is shown having a head 72 with a slot 74 and an elongated threaded portion 76 for attachment to a human bone, generally being a pedicle of the spinal column. A rod 78 is shown in position above the pedicle screw 70 ready for attachment thereto. A set screw 80 is shown having a threaded portion 82 and a break-off head portion 84. The threaded portion 82 would be received into an internally threaded portion 86 adapted to receive the threaded portion 82 of the set screw 80. The break-off head portion 84 is hexagonally shaped and would mate with the internal hexagonally shaped configuration 24 of the distal tip 22 of the inner shaft 12. The pedicle screw 70 shown is a multiaxial head pedicle screw as is well known in the art; however this could be a fixed head pedicle screw or any other bone screw where it is important to affix a longitudinal rod to the screw to maintain the position of various bone segments together. Furthermore, the set screw break-off portion 84 and rod reducer 10 have a hexagonal configuration; however, this could also be any convenient shape to provide an optimum rotational driving force to install the bone screw into a bone segment. It is important only that the shape of the portion 84 and internal driver configuration 24 be of a matching configuration. FIG. 3 shows the rod reducer 10 with the distal tip 22 of the inner shaft 12 extending distally beyond the distal 36 end of the outer shaft 30. The set screw 80 would be positioned within the inner shaft 12 such that the break-off portion 84 would be captured within the axial bore 18 with the fingers 26 lightly grasping the side walls of the break-off head portion 84. The outer shaft 30 would then be released to allow it to move to the balanced or at rest position shown in FIG. 4.

Referring to FIG. 4, the rod reducer 10 is shown in the balanced position with the set screw 80 shown captured within the rod reducer. The outer shaft 30 of the rod reducer 10 has a distal end 36 having a generally square configuration 100. The distal end has four prongs 102 extending distally from the outer shaft 30. It may be desirable to have two relatively flat surfaces 104 and 106 which are diametrically opposed to one another and two somewhat rounded surfaces 108 and 110 which are diametrically opposed to one another. However, this is not strictly necessary and in fact all surfaces 104, 106, 108 and 110 may be flat or all somewhat rounded. As shown in FIG. 4, the flat surfaces 104 and 106 each have a generally rounded slot 112 provided between prongs 102. These rounded slots 112 will act to contact the rod 78 when the rod reducer 10 is in use. The generally rounded surfaces 108 and 110 each have a narrow slot 116 provided between prongs 102. The slots 116 will allow for the use of a rod forceps or rocker tool 90 as discussed below. The inside configuration of the distal end 36 is designed to closely fit about the head portion 72 of the pedicle screw 70 such that the head portion 72 is unable to rotate with respect to the distal end 36 of outer shaft 30. Furthermore, due to the close fit about the head of the pedicle screw the axial center of the threaded portion 86 of the head portion 72 would be generally coaxial with the axial center of the inner and outer shafts 12 and 30.

Referring to FIG. 4, the rod 78 is shown in position slightly above the pedicle screw 70 ready to be fully reduced into the slot 74 of the head 72 of the pedicle screw 70. It may be desirable to use a rod forceps or rocker tool 90, a device known in the industry and discussed more fully in U.S. Pat. No. 6,036,692, previously incorporated herein by reference. Typically such an instrument is provided with a bifurcated gripping nose on one end adapted to pivotally engage a pedicle screw while the rod passes between the branches of the bifurcated gripping nose. The forceps have defined a fulcrum adjacent the gripping nose. This pivotally engaging gripping nose and included fulcrum permit a surgeon user to urge the rod into a position adjacent the slot in the pedicle screw while securely gripping the pedicle screw. The gripping nose has a pair of cylindrical projections 92 which when used would grasp the head 72 of a pedicle screw 70 as shown in FIG. 4. It is desirable for the rod reducer 10 of the present invention to be able to be used with such a rocker tool 90 to facilitate the operation of moving the rod 78 within the slot 74 of the pedicle screw 70. Typically the rocker tool 90 would be used to maneuver the rod within close approximation to the slot of the pedicle screw. Then, the rod reducer 10 would be used to fully seat the rod 78 into the slot 74 of the pedicle screw 70.

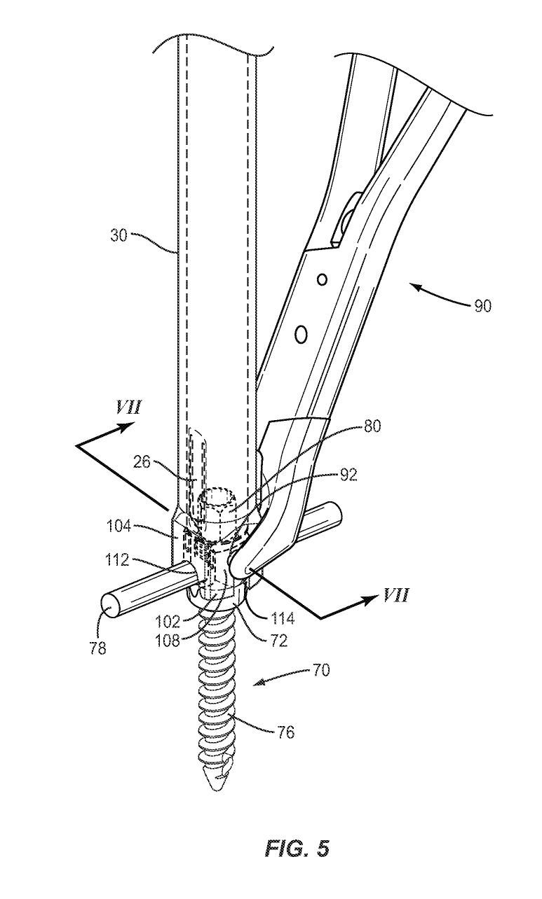

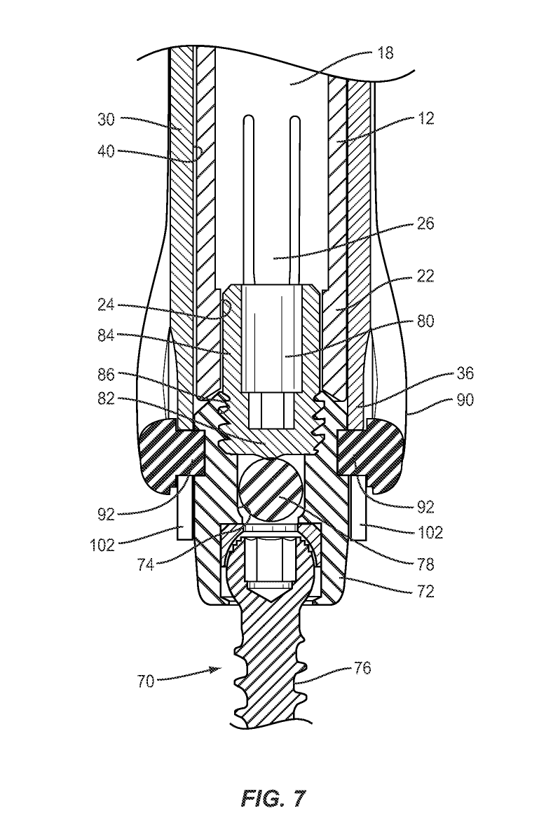

Referring to FIGS. 5 and 7, the rod reducer 10 is shown being used in conjunction with the rocker tool 90 to fully seat the rod 78 into the slot 74 of the pedicle screw 70. The surgeon user would grip the enlarged knob 32 of the outer shaft 30 and urge the distal end 36 against the rod 78 to fully seat the rod 78 in the slot 74 of the pedicle screw 70. The surgeon user may also be gripping the rocker tool 90 against the rod to assist in this function. In either case, the prongs 102 of the distal end 36 of the outer shaft 30 will slide over the head 76 of the pedicle screw 70 to allow the rounded slots 112 of the distal end 36 to urge the rod 78 into the slot 74 of the pedicle screw 70 and the narrow slots 116 of the distal end 36 will slide about the rounded projections 92 of the rocker tool 90. This allows the surgeon user to fully seat the rod into the slot of the pedicle screw with the rod reducer 10 while also positively gripping the head portion 72 of the pedicle screw with the rocker tool 90. The rocker tool 90 may now be removed from the head of the pedicle screw. The surgeon user will now want to thread the set screw 80 into the internally threaded portion 8 of the head of the pedicle screw 70 to capture the rod 78 within the slot 74 of the pedicle screw 70. While gripping the enlarged knob 32 of the outer shaft 30 the surgeon user will also grasp the handle 14 of the inner shaft 12. As shown in FIG. 5 the set screw 80 is held within the distal tip 22 of the inner shaft 12. The surgeon user will apply axial force on the inner shaft 12 with respect to the outer shaft 30 in a distal direction to allow the threaded portion 82 of the set screw to contact the head portion 72 of the pedicle screw 70. By rotating the inner shaft 12 with respect to the outer shaft 30 the surgeon user will start threading the set screw into the head of the pedicle screw. The surgeon user is also urging the outer shaft 30 against the rod 78 to fully seat the rod into the slot of the pedicle screw. It should be noted that since the prongs 102 of the outer shaft 30 are closely received about the head portion 76 of the pedicle screw, the head portion is prevented from any rotation with respect to the outer shaft 30. Furthermore, since the axial center of the internally threaded portion 86 of the pedicle screw 70 is now generally coaxial with both of the inner and outer shafts 12 and 30 of the rod reducer 10, the surgeon user can easily start threaded the set screw 80 into the head portion 72 of the pedicle screw 70. The present red reducer 10 assists in preventing any cross threading between the set screw and pedicle screw.

Referring to FIGS. 5 thru 7, the surgeon user will continue to rotate the inner shaft 12 of the rod reducer 10 to fully install the set screw within the slot 74 of the pedicle screw 70. Once the set screw contacts the rod it will become significantly harder to continue rotation of the inner shaft 12 and set screw 80. The set screw 80 is designed to separate between the threaded portion 82 and break off head portion 84 at a predetermined rotational force. However, this still requires the surgeon user to exert a significant rotational force on the inner shaft 12 and captured set screw and it is important that this rotational force not be exerted on the head portion 72 of the pedicle screw 70. This counter torque feature is accomplished by the surgeon user holding the enlarged knob 32 of the outer shaft 30 steady while only rotating the inner shaft 12. The distal end 36 of the outer shaft 30 firmly holds the head of the pedicle screw against any rotational movement while allowing the inner shaft 12 and set screw 80 to be threaded into the head of the pedicle screw until such time and the break off head portion 84 separates from the threaded portion 82 of the set screw 80. The fingers 26 of the inner shaft 12 will hold the break off head portion 84 within the confines of the rod reducer 10 until the instrument is removed from the surgical site so as to prevent the possibility of it falling into the wound site. The break off head portion 84 can be removed from the inner shaft 12 by either banging the proximal end of the handle 14 against a table, or the like, or by inserting on object into the distal tip 22 of the inner shaft to cause the break off head portion 84 to travel down axial bore 18 to opening 20 to exit the rod reducer. When installing multiple set screws, the surgeon user would simply install a second set screw into the inner shaft which would push the previous broken off break off head portion farther up into the axial bore of the inner shaft 12. This previously broken off portion could then be immediately removed through opening 20 or simply left inside the axial bore until the second set screw is installed within a second pedicle screw thereby causing a second break off head portion of the second set screw being captured by fingers 26 within the axial bore 18 of the inner shaft 12. This operation could be completed multiple times until the rod has been fully connected to the multiple pedicle screws utilized in the surgical operation.

Yet, another feature provided by the present rod reducer is its ability to prevent head splay of the head portion 72 while installing the set screw 80 into the slot 74. Over the years in this type of pedicle screw it has been known to have the set screw pop out of the slot of the pedicle screw while threading the set screw into the slot 74 to capture the rod 78 within the pedicle screw 70. Both fixed head and multiaxial head pedicle screws are typically provided with slots to receive a rod therein. However, the slot is formed by the pedicle screw having two arms which are typically internally threaded 86 and if the surgeon user over threads the set screw into the slot it is possible that the sides of the slots actually bend outwardly slightly allowing the set screw to “pop out” of the slot. In this situation, the pedicle screw should be removed and a new pedicle screw installed in its place. This is both costly and time consuming for the surgeon user. The present rod reducer 10 has the four prongs 102 which slide about the head portion 72 of the pedicle screw as discussed above. These prongs 102 assist in preventing the head portion 72 bordering the slot 74 from expanding outwardly when the inner shaft 12 is being rotated to install the set screw 80 within the slot 74. The prongs 102 of the distal end 36 of the outer shaft 30 are held still against rotation while rotating the inner shaft 12 so that the rod reducer 10 is both a counter torque and anti-head splay prevention instrument.

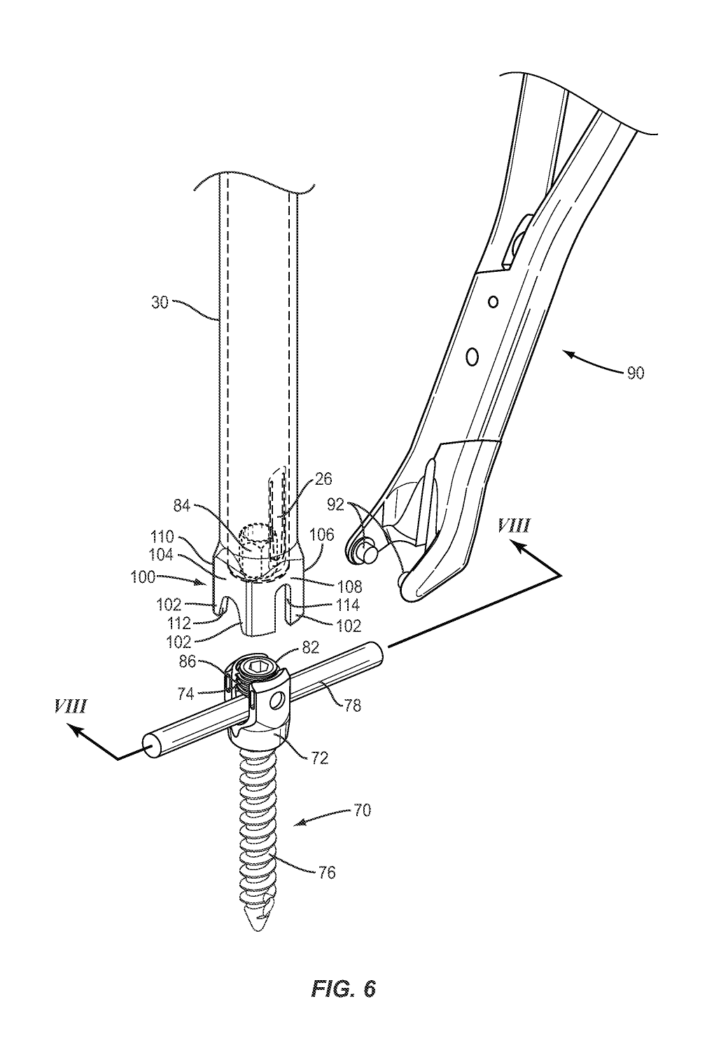

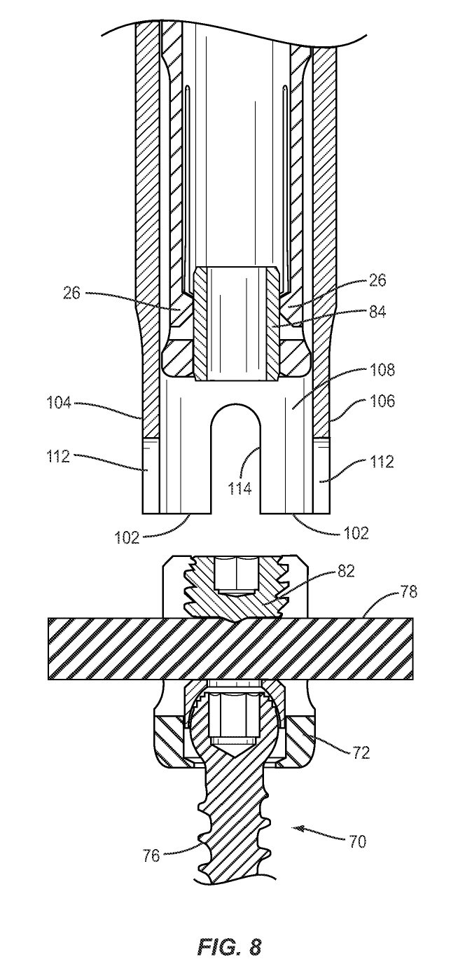

Now referring to FIGS. 6 and 8, the rod reducer 10 and rocker tool 90 are shown separated from the pedicle screw 70, rod 78 and threaded portion 82 of the set screw of the spinal construct. The threaded portion 82 of the set screw 80 is shown firmly capturing the rod 78 within the slot 74 of the head portion 72 of the pedicle screw 70. The break off head portion 84 of the set screw 80 is shown captured by the fingers 26 within the axial bore 18 of the inner shaft 12. FIG. 6 only shows one pedicle screw being attached to the rod; however, in an actual spinal construct there would be multiple pedicle screws attached to the rod.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. Any theory, mechanism of operation, proof or finding stated herein is meant to further enhance understanding of the present invention, and is not intended to make the present invention in any way dependent upon such theory, mechanism of operation, proof or finding.

It should be understood that while the use of the word preferable, preferably or preferred in the description above indicates that the feature so described may be more desirable, it nonetheless may not be necessary, and embodiments lacking the same may be contemplated as within the scope of the application, that scope being defined by the claims that follow. In reading the claims, it is intended that when words such as “a”, “an”, “at least one”, and “at least a portion” are used, there is no intention to limit the claim to only one item unless specifically stated to the contrary in the claim. Further, when the language “at least a portion” and/or “a portion” is used, the item may include a portion and/or the entire item unless specifically stated to the contrary.

What is claimed is:

1. A surgical instrument for reducing a rod toward a bone fastener and securing it thereto with a set screw, comprising:
a cylindrical fastener engaging member extending between a proximal end and a distal end, a distal portion of said fastener engaging member being adapted to receive a portion of the rod therein and being further adapted to engage the bone fastener;
a coaxial set screw installation member extending between a proximal end and a distal end, the distal end being adapted to receive and hold the set screw securely thereto; and
the fastener engaging member and coaxial set screw installation member being operable together to reduce the rod distally into the bone fastener and then rotationally attach the set screw into the bone fastener while holding the rod in contact with the bone fastener.

2. The instrument of claim 1, wherein said cylindrical fastening member includes a distal portion having a plurality of distally extending prongs configured to slide over the bone fastener to hold said bone fastener securely within said distal portion such that the bone fastener is unable to rotate with respect to said fastening member, the prongs further defining a pair of arcuate surfaces to capture the rod between such surfaces and the bone fastener.

3. The instrument of claim 2 for use with a break off set screw having a threaded portion for connection to the bone fastener and a break off head portion for driving the set screw in a rotational manner, wherein said coaxial set screw installation member is adapted to receive the break off head portion within its distal end, the distal end further comprising a finger spring for securely holding the set screw with respect to the set screw installation member.

4. The instrument of claim 3, wherein said coaxial set screw installation member is provided with an axial bore communicating between its distal end and a radially extending opening provided adjacent its proximal end, the axial bore adapted to provide a channel for receiving the break off head portion of the set screw and communicating said break off head portion exteriorly from said instrument.

5. The instrument of claim 3, wherein said coaxial set screw installation member further having a relatively uniform cylindrical shaft having an enlarged cylindrical portion spaced between its proximal end and distal end, and said cylindrical fastener engaging member having a first distal bore slightly larger than the cylindrical shaft of the set screw installation member and a second proximal bore slightly larger than the enlarged cylindrical portion of said set screw installation member, the cylindrical fastener engaging member having external threads on its proximal end receiving a nut positioned about the uniform cylindrical shaft between the enlarged cylindrical portion of the set screw installation member and its proximal end;
a pair of springs being provided about the uniform cylindrical shaft of the set screw installation member on either side of the enlarged cylindrical portion thereof and captured within the second proximal bore of the cylindrical fastener engaging member between the nut and the first distal bore thereof, to capture the fastener engaging member and set screw installation member together in a coaxial relationship in an at rest position; and
the fastener engaging member and set screw installation member being movable with respect to one another to allow the fastener engaging member to engage the rod to reduce it to the bone fastener and then to allow the set screw installation member to rotationally install the set screw into the bone fastener while simultaneously reducing the rod to the bone fastener.

6. The instrument of claim 2 for use with a rod rocker tool, wherein the distal portion of said fastener engaging member further comprises a pair of slots offset 90 degrees from said pair of arcuate surfaces for receiving a portion of the rod rocker tool such that the rod rocker tool can be utilized to hold the bone fastener while engaging the fastener engaging member to the rod and bone fastener.

7. The instrument of claim 3, wherein the finger spring comprises a pair of finger springs positioned about the distal end of the coaxial set screw installation member.

8. The instrument of claim 7, wherein the pair of finger springs are positioned 180 degrees apart from one another about the distal end of the coaxial set screw installation member.

9. A surgical instrument for reducing a rod toward a bone fastener having a head and bone threads, the head having a slot for receiving a rod and set screw for securing the rod to the bone fastener, the surgical instrument comprising:
an inner shaft extending between a proximal end and a distal end, the proximal end having a handle thereon for grasping by a surgeon user, the distal end having a receiving portion to receive and hold the set screw for rotational attachment to the bone fastener;
an outer shaft extending between a proximal end and a distal end, the outer shaft having a first axial bore receiving the inner shaft in a coaxial manner, the proximal end having an enlarged knob for grasping by a surgeon user, the distal end having an arcuate surface for engaging a portion of the rod therein and being configured to capture the head of the bone fastener; and
wherein the outer shaft is operable to reduce the rod into the slot of the bone fastener and to grasp the head of the bone fastener to prevent any rotational movement of said bone fastener and the inner shaft is operable to rotationally attach the set screw to the head of the bone fastener to affix the rod to the bone fastener.

10. The instrument of claim 9, further comprising:
the inner shaft having an enlarged cylindrical portion spaced between its proximal and distal ends, a first spring positioned about said inner shaft proximal to said enlarged portion and a second spring positioned about said inner shaft distal to said enlarged portion;
the outer shaft having a second axial bore proximal to said first axial bore, the second axial bore having a diameter slightly larger than the diameter of the enlarged cylindrical portion of said inner shaft, the distal end of the outer shaft having threads provided thereon;
a nut provided about the inner shaft and positioned between said enlarged portion and the distal end, the nut having threads operable for connection of the nut to the distal end of the outer shaft, the nut capturing the first and second springs within the second axial bore of the outer shaft under compression such that the outer shaft is held in an at rest position with respect to the inner shaft; and
whereby when the outer shaft and inner shaft are held in the at rest position the distal end of the outer shaft extends distally farther than the distal end of the inner shaft, and the outer shaft can be moved axially with respect to the inner shaft in both the proximal and distal directions against the spring force created by the first and second springs contained within the second axial bore of the outer shaft to either expose the distal end of the inner shaft or allow for reduction of the rod toward the bone fastener.

11. The instrument of claim 10 for use with a set screw having a threaded portion and a break off drive portion to allow the set screw to be threaded into the slot of the bone fastener to capture the rod within the slot thereof, the break off drive portion separating from the threaded portion upon a predetermined rotational force being exerted thereon, the instrument further comprising:
the inner shaft having an axial bore extending from the set screw receiving portion of the distal end to a radial extending opening provided adjacent the handle on the proximal end thereof;
a retaining finger provided on the inner shaft adjacent the set screw receiving portion to temporarily hold the break off drive portion of the set screw to allow for rotational attachment of the set screw to the bone fastener by rotation of the inner shaft with respect to the outer shaft; and
whereby once the set screw is fully installed within the bone fastener and the break off drive portion has separated from the threaded portion the retaining finger of the inner shaft will retain the break off drive portion of the set screw within the axial bore on the inner shaft, and upon insertion of a second set screw within the set screw receiving portion of the distal end of the inner shaft the break off drive portion of the separated first set screw will be communicated proximally within the axial bore of the inner shaft until it is removed from said axial bore through the radial extending opening thereof to be removed from said instrument.

12. The instrument of claim 11, wherein the retaining finger is a pair of retaining fingers diametrically opposed to each other to grasp the break off drive portion of the set screw.

13. The instrument of claim 10, wherein the distal end of the outer shaft has prongs extending distally therefrom to capture the head of the bone fastener, the outer shaft being operable by use of said enlarged knob to move toward the rod and bone fastener such that the arcuate surfaces of the distal end will contact the rod, and the prongs of the distal end will capture the head of the bone fastener to allow the rod to be inserted within the slot of the head of the bone fastener while the prongs hold the head of the bone fastener against rotational movement with respect to the inner shaft of said instrument.

14. The instrument of claim 13 for use with a rod rocker tool for further grasping the head of the bone fastener, the rod rocker tool having a pair of cylindrical projections which can be used to grasp the head of the bone fastener, the instrument further comprising:
the distal end of the outer shaft having a pair of arcuate surfaces for engaging the rod and a pair of slots offset 90 degrees from said pair of arcuate surfaces for receiving the cylindrical projections of the rod rocker tool such that the rod rocker tool can be utilized to hold the bone fastener while engaging the prongs of the distal end of the outer shaft and arcuate surfaces of the distal end of the outer shaft to the bone fastener and rod to seat the rod within the slot of the bone fastener.

15. A method of reducing and connecting an elongated rod to a bone fastener with a set screw in orthopedic surgery, comprising:
fixing a bone fastener to a patient;
placing an elongated rod adjacent said bone fastener;
providing a rod reducer instrument having a cylindrical fastener engaging member extending between a proximal end and a distal end, the proximal end having an enlarged knob, and the distal end having a rod receiving portion and a bone fastener receiving portion, and further having a coaxial set screw installation member extending between a proximal end and a distal end, the proximal end having a handle, and the distal end having a set screw receiving portion, the bone fastener receiving portion adapted to hold the bone fastener against rotation with respect to the coaxial set screw installation member;
inserting a set screw into the set screw receiving portion of the coaxial set screw installation member;
placing the distal end of said fastener engaging member of said rod reducing and connecting instrument over the elongated rod and bone fastener such that the rod receiving portion is contacting the elongated rod and the bone fastener receiving portion is contacting the bone fastener and reducing the rod toward the bone fastener by use of the enlarged knob;
urging the distal end of the coaxial set screw installation member toward the bone fastener until the set screw in contacting the bone fastener and rotating the coaxial set screw installation member by use of the handle to rotationally affix the set screw into the bone fastener to capture the elongated rod to the bone fastener.

16. A method of claim 15 for use with a break off set screw having a threaded portion for connection to the bone fastener and a break off drive portion for driving the set screw in a rotational manner, wherein said coaxial set screw installation member is adapted to receive the break off drive portion within its distal end, the distal end further comprising a finger spring for securely holding the set screw with respect to the set screw installation member, further comprising:
rotating the handle of the coaxial set screw installation member to rotationally affix the set screw to the bone fastener to capture the elongated rod to the bone fastener until the break off drive portion separated from the threaded portion of said set screw; and
removing the rod reducer instrument from the orthopedic surgery site, the finger spring securely holding the break off drive portion of the set screw within the set screw installation member.

17. A method of claim 16 wherein the finger spring comprises a pair of finger springs positioned about the distal end of the coaxial set screw installation member.

18. A method of reducing and connecting an elongated rod to a bone fastener with a set screw in orthopedic surgery, comprising:
fixing a bone fastener to a patient;
placing an elongated rod adjacent said bone fastener;
providing a rod rocker instrument about the elongated rod and grasping the bone fastener;
providing a rod reducer and connection instrument having a cylindrical fastener engaging member extending between a proximal end and a distal end, the proximal end having an enlarged knob, and the distal end having a rod receiving portion and a bone fastener receiving portion, and further having a coaxial set screw installation member extending between a proximal end and a distal end, the proximal end having a handle, and the distal end having a set screw receiving portion, the bone fastener receiving portion adapted to hold the bone fastener against rotation with respect to the coaxial set screw installation member, the bone fastener receiving portion further having slots for sliding about the rod rocker instrument grasping the bone fastener;
inserting a set screw into the set screw receiving portion of the coaxial set screw installation member;
placing the distal end of said fastener engaging member of said rod reducing and connecting instrument over the elongated rod and bone fastener such that the rod receiving portion is contacting the elongated rod and the bone fastener receiving portion is contacting the bone fastener and reducing the rod toward the bone fastener by use of the enlarged knob while holding the bone fastener with the rod rocker instrument, the slots of the bone fastener receiving portion sliding about the rod rocker tool grasping the bone fastener;
urging the distal end of the coaxial set screw installation member toward the bone fastener until the set screw in contacting the bone fastener; and
rotating the coaxial set screw installation member by use of the handle to rotationally affix the set screw into the bone fastener to capture the elongated rod to the bone fastener.

19. A method of claim 18, for use with a break off set screw having a threaded portion for connection to the bone fastener and a break off drive portion for driving the set screw in a rotational manner, wherein said coaxial set screw installation member is adapted to receive the break off drive portion within its distal end, the distal end further comprising a finger spring for securely holding the set screw with respect to the set screw installation member, further comprising:
rotating the handle of the coaxial set screw installation member to rotationally affix the set screw to the bone fastener to capture the elongated rod to the bone fastener until the break off drive portion separates from the threaded portion of said set screw; and
removing the rod reducer instrument from the orthopedic surgery site, the finger spring securely holding the break off drive portion of the set screw within the set screw installation member.

20. A method of claim 19, wherein the finger spring comprises a pair of finger springs positioned about the distal end of the coaxial set screw installation member.
